(19) United States
(12) Patent Application Publication
(10) Pub. No.: US 2010/0173328 A1
(43) Pub. Date: Jul. 8, 2010

(54) Apparatus for measuring a cell number and a quantity of a cellular protein expression and the method thereof

(75) Inventors: Huang Haw-Ming, Taipei City (TW); Lin Jiun-Yan, Taipei City (TW); Lin Yung-Sheng, Sloushuel Township (TW)

Correspondence Address: Haverstock & Owens, 162 North Wolfe Road, Sunnyvale, CA, 94086, US

(73) Assignee: National Applied Research Laboratories and Taipei Medical University

(21) Appl. No.: 12/319,474

(22) Filed: Jan. 7, 2009

(51) Int. Cl. G01N 33/53; C12M 1/00

(52) U.S. Cl. 435/7.2; 435/287.2

(57) ABSTRACT

The apparatus and method thereof for harmlessly and continuously measuring and recording a target protein expression and a number of growing cells are provided. By causing an AC current to flow through an electrode where cells grows thereon, the target protein expression and the number of growing cells are obtained via converting the impedance values of the electrode.

### FIELD OF THE INVENTION

The present invention relates to an apparatus for measuring a cell number and an expression of a cellular protein and the method thereof, and more particularly to an apparatus for continuously and nondestructively measuring cell numbers of an attached cell or a suspended cell and an expression of a specific cellular protein and the method thereof.

### BACKGROUND OF THE INVENTION

There are various apparatuses and methods for continuously measuring behaviors of cells. However, since some of those apparatuses and methods are harmful to cells, it is difficult for long term measuring and monitoring a sample cell via those apparatuses and methods. Accordingly, an apparatus, or a method thereof, for continuously and nondestructively measuring and monitoring some parameters of cells is a demand for a laboratory and/or the biochemical industry. Nevertheless, presently, the apparatus which can continuously and nondestructively measuring and monitoring the states of cells is rare, and the operations of those apparatuses still have many limitations.

In 1984, Giaever and Keese published a method, namely Electrical Cell Impedance Spectroscopy (ECIS), for monitoring the behavior of the attached cell and continuously measuring the capacitive reactance value and the resistance value of an electrode that the attached cell is grown thereon. They discovered there is no influence on the attachment, the extension and the growth of the attached cell under the situations that the cell grows on a gold electrode or imposes an electric field on the gold electrode. Moreover, according to the measuring records, the total electric resistance was increased corresponding to the growth of the attached cell.

With regard to ECIS measuring method, since the cell membrane, like a capacitor, has the property of isolating the DC and coupling the AC, the currents used therein for measuring some parameters of the cell must be an AC signal. In many conventional methods for analyzing the impedance of the cell, a current lower than 1 μA is usually used for measurement, and a minor voltage across the two measured electrodes can be further analyzed by the user. Since the current through the measuring circuit is lower than 1 μA, the measuring time thereof is very short and it is stated in some references that the measuring steps in those methods will not influence or change the physiology of the cell, those methods can be referred to nondestructively measuring methods.

In recent studies, ECIS measuring method is specifically used for monitoring and measuring the cell numbers of the attached cells including the fibroblast, the endothelial cell, the astrocyte, the kidney cell, the hepatoma cell and the Hela cell. Those experimented attached cells can be the cell line and the primary culture. However, ECIS measuring method still cannot be used for measuring the cell number of the suspended cell.

With regard to the designs of the electrode used in ECIS measuring method, most of which are gold electrodes and are configured on plastic culture dishes. Moreover, the electrodes on one culture dish include a minor detecting electrode and a major counting electrode. According to the references, it is disclosed that the changes of the cell cannot be measured if the two electrodes, i.e. the detecting electrode and the counting electrode, have the same area. However, some scholars are successful to continuously monitor and measure the behaviors of the attached cell by interdigitated electrodes (IDEs) made of the platinum. Based on the above, it is shown that the design and the area of the electrodes will influence the feasibilities of measurements when measuring the impedance of the electrodes.

In many studies, various designs of the electrode are tested for measuring the impedance of the cell. However, there is no solution, for measuring the cell number of the suspended cell and the quantity of a cellular protein, being provided.

Presently, the methods for measuring the cellular protein expression are roughly classified into two groups, in one of which the morphology of the sample cell will be destroyed, and in another one the morphology of the sample cell will be maintained. Both of the two methods for measuring the cellular protein expression are respectively introduced as follows.

In the first method for measuring the cellular protein expression, generally, the cell will be lysed and the proteins thereof can subsequently be isolated and purified for the qualitative and/or the quantitative analyses. Moreover, the lysates of the cell are the materials for the detection of the protein microarray or purifying the RNA therefrom for analyzing the protein expression on RNA level. After the cell being lysed, there are various methods can be utilized for the qualitative and/or the quantitative analyses of proteins. For example, the expressions of proteins can be quantitated by Western blot after the lysates of the cell being separated by a SDS-PAGE. In addition, the protein of the cell can be analyzed by mass spectrometry (MS) after a HPLC process. Moreover, an immunostain can be performed after the lysates of the cell being separated by a capillary electrophoresis (CE). However, since the sample cell in this kind of measuring method would be destroyed, in a time-course experiment, this measuring method is not an appropriate one for monitoring the sample continuously and nondestructively and is necessary to harvest the sample cell at each predetermining time.

With regard the second method for measuring the cellular protein expression, the principle thereof is taken by the immunocytochemistry and the morphology of the cell will be maintained. In detailed words, the target protein will be labeled by its antibody conjugated with the fluorescent substance. Through the immunocytochemistry, the user can observe the location of target protein in the cell by the fluorescence microscope. Moreover, the 3D conformation and the dynamic status of the target protein can be observed by the confocal microscope. By utilizing this kind of measuring method, the morphology of the cell will be maintained, however, the dish/flask cultured the cell is necessary to be removed from the incubator for observation. Accordingly, this kind of measuring method cannot be utilized in continuous observation under the microscope. In addition, measuring the protein expression by the immunocytochemistry has various defects such as needing to be operated in the dark, the decay of the fluorescent substance and the expensive experimental equipments.

The present developments of the protein biochip are introduced as follows. The protein biochips performed according to the electrical principles are mainly the immunochip. With regard to the immunochip, the working principle thereof is measuring the expression of the target protein by the specifically binding between the antigen and the antibody. In detailed words, the sample cells containing the target protein are incubated on a biochip coated with antibodies specific to the target protein, and then changes of the capacitance of the biochip can be measured. In some studies, it is shown that the measured capacitance value of the biochip will be decreased while the antigen and the antibody are bound. Sometimes, in a system, the antibodies are even conjugated on the surfaces of molecules having high electric conduction, and then the sample having the target protein, e.g. the antigen, is observed and incubated with the molecules, whereby changes of the conductivity in the system can be measured. In the mentioned system, with the increasing of the concentration of the target protein, the change of the conductivity in the system is more obvious. However, the materials incubated on the biochip is also, or is extracted from, the cell lysate. That is to say, as the conventional protein measuring methods, the sample cell must be destroyed so that a continuous and nondestructive measurement on a cell sample cannot still be achieved by the protein biochip.

Keeping the drawbacks of the prior arts in mind, and employing experiments and researches full-heartily and persistently, the applicant finally conceived apparatus for measuring a cell number and a quantity of a cellular protein expression and the method thereof.

### SUMMARY OF THE INVENTION

The present invention seeks to provide an apparatus and the method thereof for measuring a cell number and an expression of a specific protein. By causing a current flowing through the sample cell, the apparatus and method of the present invention can provide a continuous and nondestructive measurement of cell numbers of the sample cell, wherein the sample cell can be the attached or suspended cells. By adding an appropriate binder, e.g. an antibody, conjugated with a metal particle thereon into the culture medium and causing the current flowing through the sample cell, the apparatus and method of the present invention can also provide a continuous and nondestructive measurement of expressions of the specific protein.

In accordance with one aspect of the present invention, a method for continuously measuring a protein expression of a cell is provided. The method has steps of a) culturing the cell on an indium tin oxide (ITO) electrode with a medium; b) adding a first antibody specifically bound to a protein of the cell into the medium; c) adding a second antibody conjugated with a metal particle and specifically bound to the first antibody into the medium; d) causing a current flowing through the ITO electrode; e) measuring an impedance value of the ITO electrode; and f) converting the impedance value into a quantity of the protein expression by a first algorithm.

Preferably, the cell cultured in the step (a) comprises plural kinds of cells.

Preferably, the first antibody comprises a plurality of first antibodies respectively specifically bound to a plurality of proteins of the cell, and the second antibody comprises a plurality of second antibodies respectively specifically bound to the plurality of first antibodies.

Preferably, the current intermittently flows through the ITO electrode.

Preferably, the step (e) is measuring one of a capacitive reactance value and a resistance value of the ITO electrode and the step (f) is converting one of the measured capacitive reactance value and the measured resistance value into the quantity of the protein expression by a second algorithm.

In accordance with another aspect of the present invention, an apparatus for measuring a protein expression is provided. The apparatus includes a cell, a medium culturing the cell and having a binder conjugated with a metal particle and specifically bound to a protein of the cell, an electrode electrically connected with the medium, a power source electrically connected with the electrode and providing a current flowing through the electrode and a measuring unit electrically connected with the electrode and the power source, measuring a change of an impedance value of the electrode and converting the change of the impedance value of the electrode into a quantity of the protein expression by an algorithm.

Preferably, the electrode comprises two wire electrodes, each of which has a width of 0.4 mm.

Preferably, the two wire electrodes are separated from each other by a width of 4 mm.

Preferably, the electrode is made of an indium tin oxide (ITO).

Preferably, the electrode is disposed on a substrate made of one selected from a group consisting of a glass, a quartz, a plastic and a combination thereof.

Preferably, the binder comprises a first antibody and a second antibody conjugated with the metal particle and specifically bound to the first antibody, and the metal particle is a gold particle.

Preferably, the electrode is made in an array.

Preferably, the current is an alternating current.

Preferably, the apparatus further comprises a signal amplifier amplifying the change of the impedance value of the electrode. In accordance with another aspect of the present invention, a method for continuously measuring a protein expression of a cell is provided. The method has steps of a) culturing the cell on an electrode with a medium; b) adding a binder conjugated with a metal particle and specifically bound to a protein of the cell into the medium; c) causing a current flowing through the electrode; d) measuring an impedance value of the electrode; and e) converting the impedance value into a quantity of protein expression.

Preferably, the cell in step (a) is cultured on an indium tin oxide (ITO) electrode.

In accordance with another aspect of the present invention, a measuring method is provided. The measuring method has steps of a) culturing the cell on an electrode; b) causing a current flowing through the electrode; c) measuring a first parameter of the electrode; and d) converting the first parameter into a second parameter of the cell.

Preferably, the first parameter is an impedance value, the second parameter is a cell number, and the measuring method is used for continuously estimating the cell number.

Preferably, the cell cultured in the measuring method is a suspending cell and is cultured in a serum.

Preferably, the first parameter is converted into the second parameter by a algorithm.

### BRIEF DESCRIPTION OF THE DRAWINGS

FIG. 1A is a schematic diagram showing an indium tin oxide (ITO) electrode chip of the present invention; FIG. 1B is a schematic diagram showing various electrode patterns of ITO electrode of the present invention;

FIG. 2 is a schematic diagram showing the measurement of the expression of the target cellular protein by ITO electrode of the present invention;

FIG. 3 is a schematic diagram showing an apparatus of the present invention used for measuring the cell number and the expression of the target protein of the present invention;

FIG. 4 shows the differences of impedance values of cultured HL-60 cells with various initial cell seeding densities measured by the method of the present invention;

FIG. 5(A) shows the differences of impedance values of cultured MG-63 cells with various initial cell seeding densities measured by the method of the present invention; FIG. 5(B) shows the cell proliferations of MG-63 cells with various initial cell seeding densities measured by MTT (3-[4,5-Dimethylthiazol-2-yl]-2,5-diphenyl-tetrazdium bromide) method; FIG. 5(C) shows an regression analysis of the relationship between the difference of impedance values as shown in FIG. 5(A) and the O.D. values as shown in FIG. 5(B);

FIG. 6 shows the differences of impedance values of cultured MG-63 cells with various initial cell seeding densities measured by the method of the present invention;

FIG. 7 shows the fluorescein isothiocyanate (FITC) fluorescence intensities of Integrin of MG-63 cells at 48^{th} hour after seeding the cells;

FIG. 8 shows an regression analysis of the relationship between the differences of the impedance values as shown in FIG. 6 and the FITC fluorescence intensities as shown in FIG. 7; and

FIG. 9 is a diagram showing a cell culture flask 04 for obtaining the results as shown in FIG. 4 by the method of the present invention.

### DETAILED DESCRIPTION OF THE PREFERRED EMBODIMENT

In order to further illustrate the techniques, methods and efficiencies used to procure the aims of this invention, please see the following detailed description. It is believable that the features and characteristics of this invention can be deeply and specifically understood by the descriptions. It is to be noted that the following descriptions of preferred embodiments of this invention are presented herein for the purposes of illustration and description only; it is not intended to be exhaustive or to be limited to the precise form disclosed.

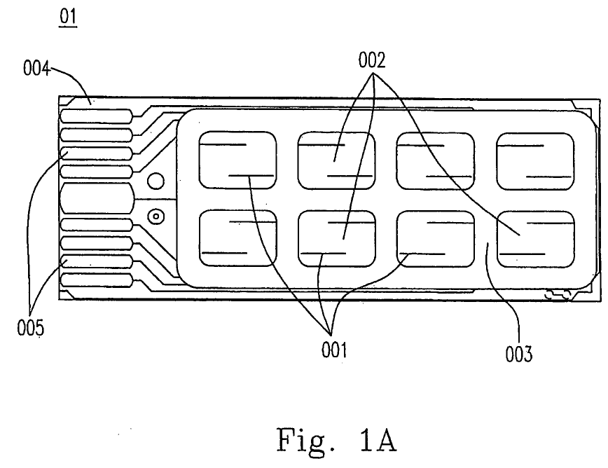

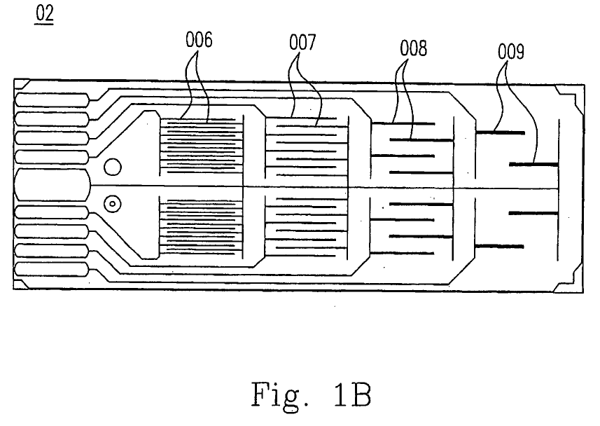

Please refer to FIG. 1A, which illustrates an indium tin oxide (ITO) electrode chip 01 on which the cell can grow normally. ITO electrode chip 01 includes an ITO electrode 001, cell culturing areas 002, a frame 003, an insulating substrate 004 and containing areas 005. Regarding with the production of ITO electrode chip 01, a specific type of ITO electrode 001 is formed on insulating substrate 004 first, and next, ITO electrode 001 is separated into various cell culturing areas 002 by frame 003. Insulating substrate 004 can be made of a glass, a quartz, a plastic, or other appropriate material and a combination thereof. For measuring the values, e.g. a cell number or a volume of a protein expression, sample cells are cultured in cell culturing areas 002 so as to make the sample cells together with the medium become a part of the sensing circuit. When the number or the impedance of the sample cell are changed, the impedance of the electrified ITO electrode chip 01 is correspondingly changed, which can be observed and/or measured from the output of containing areas 005.

The cell is a bad conductor of electricity. On the other hand, the general cell culture medium is mainly composed of the ion solution and consequently is a good conductor of electricity. When the attached cell is being the sample cell cultured in cell culturing areas 002, the attached cell would attach on ITO electrode 001 and become the circuit bridged ITO electrode 001. Therefore, change of electrical properties of the attached cell is corresponding to that of the circuit. Moreover, the attached cell would generate an impedance on ITO electrode chip 01, so that the behavior of the attached cell can be observed by measuring the impedance of ITO electrode chip 01.

In the situation of culturing a suspended cell on ITO electrode chip 01, since the suspended cell is the bad conductor of electricity and would neither contact with nor attach on ITO electrode 001, the current would flow through the culture medium and bypass the suspended cell. Accordingly, when observing the behavior of the suspended cell cultured on ITO electrode chip 01, it is necessary to replace the general culture medium by the serum. Since the resistance of the serum is higher than that of the suspended cell, the current is prompted to flow through the suspended cell. By culturing with the appropriate culture mediums, the changes of capacitive reactance value and the resistance value caused by the changes of the cell numbers of both the suspended and the attached cells can be observed and measured continuously.

In the present invention, the property of specific binding between the antigen and antibody is applied for detecting a target cellular protein and/or measuring the volume of expression of the target cellular protein. In more detail, when observing the expression of the target cellular protein by the present invention, the first antibody specifically bound to the target protein will be added into the culture medium for binding to the target proteins, and then the second antibody specifically bound to the first antibody and conjugated a metal particle thereon will be added into the culture medium for binding to the first antibody. Thus, since the differences between the electric conductivities of the metal particle, ITO electrode and the cell (where respective resistivities thereof are about 10⁻⁶ Ωcm, 2*10⁻⁴ Ωcm and 140 Ωcm), the change of the expression of the target cellular protein will be detected even if the change is very slight. Accordingly, through the present invention, a continuously observation/detection with high sensitivity for the expression of the target cellular protein is easily to achieve.

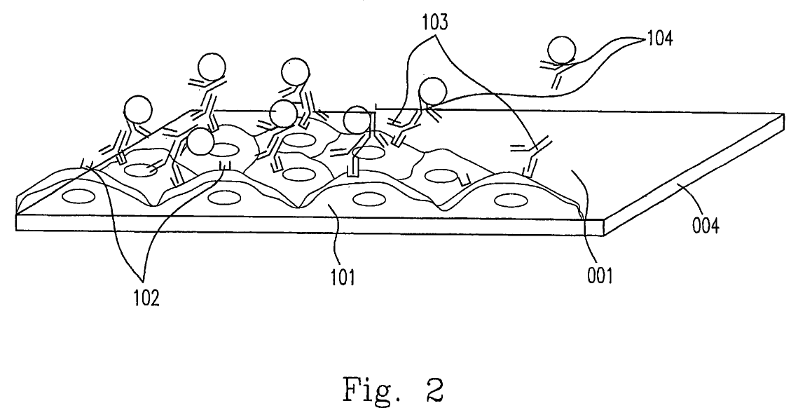

Please refer to FIG. 1B, which illustrates several electrode patterns of ITO electrode. In the present invention, the electrode patterns are varied from the interdigital electrode structure (IDES). The respective widths of electrodes 006, 007, 008 and 009 are 0.05 mm, 0.1 mm, 0.2 mm and 0.4 mm, and each width of gaps between two adjacent electrodes is ten-folds of the respective widths of electrodes. For example, each width of electrodes 009 is 0.4 mm and the width of gap between two adjacent electrodes 009 is 4 mm. After preliminary tests relative to the several electrode patterns of ITO electrode, the electrode pattern of electrodes 009 revealed better and more stable results. Accordingly, electrode patterns of ITO electrode chips applied in all the experiments of the present invention are designed as the electrode pattern of electrodes 009 shown in FIG. 2B. In addition, various values of the current flowed through the ITO electrode of the present invention are also tested for obtained the appropriate value(s) for the present method, and it is found that a value of the current lower than 1 μA applied to the present method would provide a better result of the relevantly experimental measurements.

Please refer to FIG. 2, which shows the continuous and nondestructive measurement of the expression of the target cellular protein by ITO electrode 001 as shown in FIG. 1. FIG. 2 shows ITO electrode 001, insulating substrate 004, cells 101, cellular protein 102 of cells 101, first antibody 103 specifically bound to cellular protein 102 and second antibody 104 specifically bound to first antibody 103 and conjugated a metal particle thereon.

When observing the expression of cellular protein 102 by ITO electrode 001, cells 101 need to be normally grown on ITO electrode 001 first, and then an AC current is generated to flow through ITO electrode 001 and an initial impedance value of ITO electrode 001 is can be measured. Next, first antibody 103 is added into the culture medium for binding to cellular protein 102 expressed on cells 101, and then second antibody 104 is added into the culture medium for binding first antibody 103. Since second antibody 104 is conjugated therewith the metal particle, the impedance property of cells 101 bound with second antibody 104 will be changed. In other words, the impedance value of ITO electrode 001 on which cells 101 bound with second antibody 104 grows will be different from the initial impedance value. Therefore, as the change of the expression of cellular protein 102, the volume of second antibody 104 bound to cellular protein 102 will increase accordingly. By causing the AC currents, where the values thereof are identical to that of the AC current flowing through ITO electrode 001 initially, respectively flow through ITO electrode 001 at differently predetermined times, the different impedance values of ITO electrode 001 can be measured and the expressions of cellular protein 102 can be conversed thereby. For example, the expressions of cellular protein 102 can be conversed from the different impedance values of ITO electrode 001 by an algorithm. In a preferable embodiment, the AC current flows through the ITO electrode in an intermittent form, whereby the possible inferences causing by the AC current flowing through the sample cells will further be decreased.

In a preferable embodiment, cells 101 in FIG. 2 can include more than two different kinds of cells. By the present method or apparatus, plural kinds of cells are incubated on ITO electrode chip 01 and the interactions among the plural kinds of cells and the specific protein(s) can be easily observed. In such experiments, first antibody 103 may include more than two kinds of antibodies for respectively binding to the proteins of the plural kinds of cells, where the contain of second antibody 104 is adjusted accordingly, if necessary. Moreover, based on the similar concepts, the present method or apparatus is also applicable to observe the interactions among plural kinds of target proteins of the same cell, which can be achieved by adjusting the contains of first antibody 103. For example, if Protein A and Protein B are expressed by the sample cell, it can be demonstrated whether the expression of a Protein A will increase the expression of a Protein B by appropriate first antibody 103, second antibody 104 and control experiments of the method of the present invention.

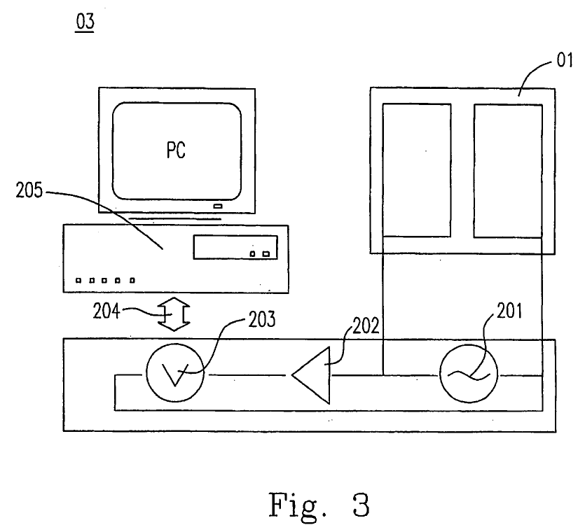

Please refer to FIG. 3, which shows an apparatus 03 of the present invention used for measuring the cell number and the expression of the target protein. Apparatus 03 includes ITO electrode 001, power source of AC current 210, signal amplifier 202, signal collector 203, recording and controlling interface 204 and computer 205, and all of which are electrically connected to each other.

Please still refer to FIG. 3. When observing the expression of the target cellular protein by apparatus 03, the sample cells (not shown) need to normally grow on ITO electrode 001 first, where the culture medium (not shown) is added with a binder, e.g. an antibody, conjugated with a metal particle and specifically bound to the target cellular protein. By providing a steadily slight AC current being harmless for the sample cell to ITO electrode 001 where the sample cells grow thereon at various but continuous times, potential differences of ITO electrode 001 can be measured. The measured potential differences will amplify by signal amplifier 202 and collect by signal collector 203. By recording and controlling interface 204 and computer 205, the change of the values of the measured potential differences can be observed, whereby the expressions of the target cellular protein and the change thereof can be converted and obtained.

Apparatus 03 shown in FIG. 3 is also applicable to observe the proliferation of the sample cells. As mentioned above, since the respective resistivities of ITO electrode and the cell are different from each other, the sample cells grown on ITO electrode 001 will cause the change of the impedance property of ITO electrode 001. Thus, the cell numbers of the sample cells at various times are easily to be obtained by apparatus 03.

In a preferable embodiment, ITO electrode 001 which the sample cells are cultured thereon needs not to take out from the incubator. By apparatus 03, it is convenient for the user to observe and measure the growth of the sample cells continuously and nondestructively if only the mentioned units of apparatus 03 are electrically connected to ITO electrode 001 in the incubator. It is very advantageous that the sample cells can be observed in a stable environment and conditions since the sample cells need not to leave from the incubator during the experimental period.

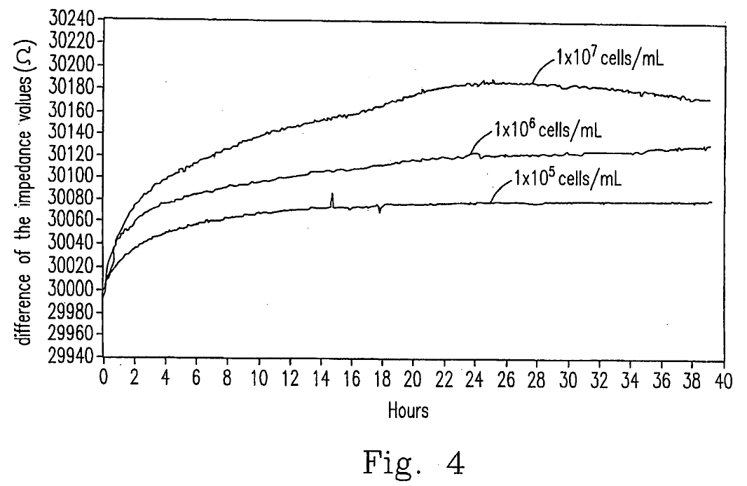

Please refer to FIG. 4, which shows the record and measurement of cell numbers of the suspended cell, i.e. HL-60 cell line (BCRC No. 60027), which is obtained by apparatus 03 of the present invention. The culture medium for culturing HL-60 cells is the fetal bovine serum. In the relevant experiments of FIG. 4, three initial cell seeding densities, 1*10⁵ cells/mL, 1*10⁶ cells/mL and 1*10⁷ cells/mL, are respectively seeded on cell culturing areas 002 of ITO electrode chip 01, and HL-60 cells on ITO electrode 001 are cultured in the incubator under the conditions of 5% CO₂ and 37° C. The X-axis of FIG. 4 shows the experimental time and the unit thereof is the hour, and the Y-axis of FIG. 4 shows the differences of impedance values between the control group and the respective experimental groups and the unit thereof is the ohm (Ω), where the differences of impedance values mentioned hereinafter are under the definition the same as that of FIG. 4. In detail words, the control group is observing the change of a cell culturing area 002 contains the fetal bovine serum without HL-60 cells being seeded therein, and the experimental groups are observing the changes of cell culturing areas 002 each of which is seeded with the respective initial cell seeding densities namely 1*10⁵ cells/mL, 1*10⁶ cells/mL and 1*10⁷ cells/mL. In the relevant experiments of FIG. 4, an intermittent AC current flows through the ITO electrode for 800 millisecond (ms) and is paused for 5 minutes, where the intermittent AC current would be provided and paused according to the mentioned frequency until the end of the experiment. In addition, the intermittent AC current has a steady value of 1 μA. When the AC current flows through cell culturing areas 002, i.e. ITO electrodes 001, of the control and experimental groups, the respective impedance values of those ITO electrodes 001 are generated and measured by apparatus 03, and differences of impedance values between the control group and the respective experimental groups are obtained. With regarding the data shown in FIG. 4, the differences of impedance values is measured and calculated per 5 minutes.

As shown in FIG. 4, it is known that the higher initial cell seeding density of cells are seeded, the greater differences of impedance values between control and experimental groups are observed; the impedance values of the respective experimental groups are increased follow the proliferations of the suspended cell, HL-60. On the other hand, it is also applicable to seed the cells with various initial cell seeding densities on respective ITO electrode chips, or to seed the cells with an identical initial cell seeding density in respective cell culturing areas 002 of an ITO electrode chip for getting the mentioned data.

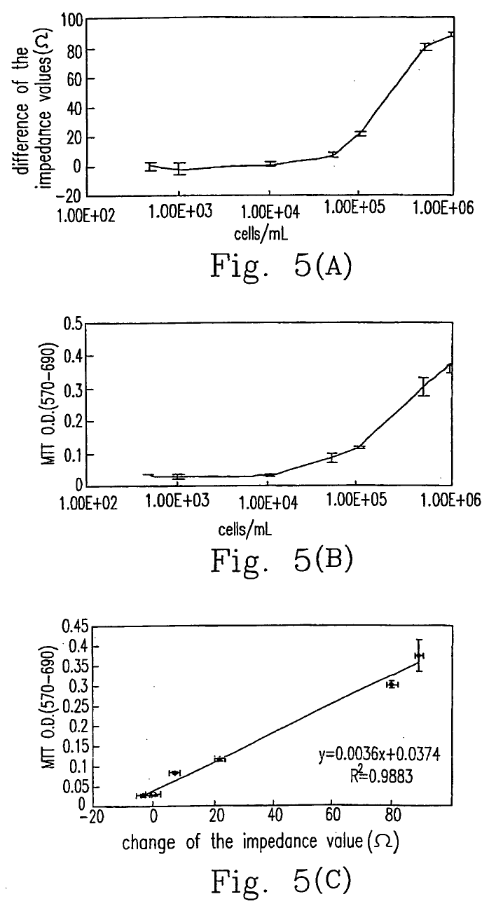

Please refer to FIG. 5(A), which shows the record and measurement of cell numbers of an attached cell, i.e. MG-63 cell line (BCRC No. 60279), which is obtained by apparatus 03 of the present invention. The culture medium for culturing MG-63 cells is the high glucose DMEM with 10% fetal bovine serum and 1% antibiotic, and MG-63 cells on ITO electrode 001 are cultured in the incubator under the conditions of 5% CO₂ and 37° C. The X-axis of FIG. 5(A) shows the initial cell seeding density and the unit thereof is cells/mL, and the Y-axis of FIG. 5(A) shows the differences of impedance values between the control group and the respective experimental groups and the unit thereof is the ohm (Ω). In the experiments obtaining the data of FIG. 5(A), various initial cell seeding densities of cells, MG-63, are respectively seeded in cell culturing areas 002. After 24 hours that MG-63 cells have well attached on ITO electrodes 001 in respective cell culturing areas 002, impedance values of each ITO electrode 001 of control and the experimental groups are obtained by causing an AC current having a value of 1 ΩA flowing through each ITO electrode 001 at an appropriate time. Thus, differences of impedance values between the control group and the respective experimental groups are obtained. As shown in FIG. 5(A), it is clear that the higher initial cell seeding density of cells are seeded, the greater differences of impedance values between control and experimental groups are observed.

Please refer to FIG. 5(B), which shows the cell proliferations of MG-63 cells with various initial cell seeding densities measured by MTT (3-[4,5-Dimethylthiazol-2-yl]-2,5-diphenyl-tetrazdium bromide) method, where the MG-63 cells are cultured in respective cell culturing areas 002 and in the incubator under the conditions of 5% CO₂ and 37° C. The X-axis of FIG. 5(B) shows the initial cell seeding density and the unit thereof is cells/mL, and the Y-axis of FIG. 5(B) shows the optical density (O.D.) value with a determination wavelength of 570 nm and a reference wavelength of 690 nm. In the experiments obtaining the data of FIG. 5(B), after 24 hours MG-63 cells being seeded, MTT method is applied for obtaining the respective O.D. values of MG-63 cells with various initial cell seeding densities. As shown in FIG. 5(B), it is clear that the higher initial cell seeding density of MG-63 cells are seeded, the higher cell activity will be observed.

Please refer to FIG. 5(C), which shows a regression analysis of the relationship between the difference of impedance values as shown in FIG. 5(A) and the O.D. values as shown in FIG. 5(B). The X-axis of FIG. 5(C) shows the difference of impedance values and the unit thereof is the ohm (Ω), and the Y-axis of FIG. 5(C) shows the optical density (O.D.) value with a determination wavelength of 570 nm and a reference wavelength of 690 nm. As revealed in FIG. 5(C), it is clear that, under one initial cell seeding density, the difference of impedance values and the optical density (O.D.) value, obtained by MTT method, of MG-63 cells have a highly linear correlation (R²=0.9883 and p<0.005). Moreover, it is also clear from FIG. 5(C) that the difference of impedance values is in direct proportion with the initial cell seeding density. Based on the illustrations of FIGS. 5(A) to 5(C), it is shown ITO electrode chip 01 of the present invention is suitable for continuously observing the growth and proliferation of cells and applicable for the fields of cytotoxicity assay and screening of potential drugs.

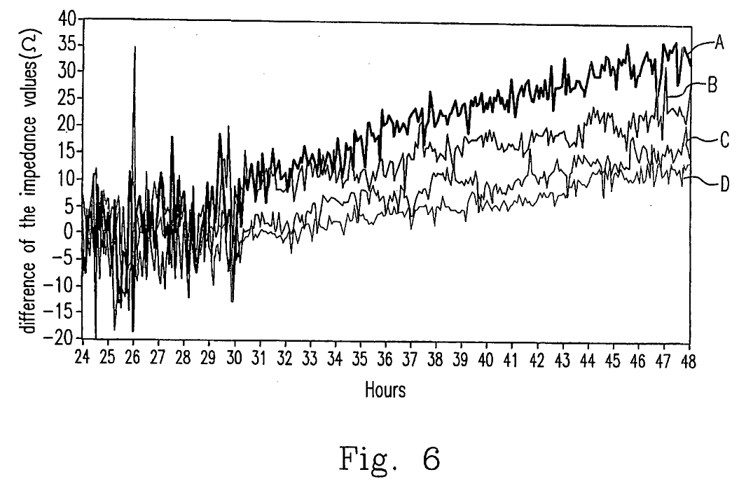

Please refer to FIG. 6, which shows the changes of the impedance values of ITO electrode caused by the Integrin, wherein the changes are measured and calculated by apparatus 03 of the present invention and revealed by curves, and the Integrin is a cellular protein continuously expressed when the cell is proliferating. Regarding the experiments obtaining the data of FIG. 6, four initial cell seeding densities of 5*10⁵ cells/mL, 2.5*10⁵ cells/mL, 1.2*10⁵ cells/mL and 5*10⁴ cells/mL of MG-63 cells are respectively seeded in cell culturing areas 002 for being the materials of the control and the experimental groups. The culture medium for culturing MG-63 cells is the high glucose DMEM with 10% fetal bovine serum and 1% antibiotic, and MG-63 cells on ITO electrode 001 are cultured in the incubator under the conditions of 5% CO₂ and 37° C. The X-axis of FIG. 6 shows the experimental time and the unit thereof is the hour, and the Y-axis of FIG. 6 shows the differences of impedance values between the control group and the respective experimental groups and the unit thereof is the ohm (Ω).

In the experiments obtaining the data of FIG. 6, after 24 hours MG-63 cells were seeded (i.e. 0-24 hours but not shown on X-axis of FIG. 6), the mouse anti-human Integrin β1 IgG antibody and the goad anti-mouse IgG-Au antibody are added into the culturing mediums of respective MG-63 cells with four different initial cell seeding densities, i.e. the four experimental groups, and PBS and the goad anti-mouse IgG-Au antibody are added into the culturing mediums of respective MG-63 cells with four different initial cell seeding densities, i.e. the four control groups. After mentioned antibodies-added procedures, all of MG-63 cells of control and experimental groups cultured on ITO electrode chip 01 of the present invention are moved into the incubator and continuously observed and measured for 24 hours by apparatus 03.

Please still refer to FIG. 6, the impedance values of each ITO electrodes 001, where the MG-63 cells of control and experimental groups are cultured thereon, are measured and recorded by apparatus 03. In the relevant experiments of FIG. 6, the intermittent AC current flows through the ITO electrode for 800 millisecond (ms) and is paused for 5 minutes, where the time frequency of the intermittent AC current will be continued until the ends of the experiments. In addition, the intermittent AC current has a steady value of 1 μA. Further, the difference of the impedance values of one of the experimental groups and its corresponding control group, i.e. the control group having the initial cell seeding density identical to the one of the experimental group, at a specific time is calculated and recorded. By collecting the mentioned differences of all the four experimental and control groups, the curves shown in FIG. 6 are obtained. In FIG. 6, curves A, B, C and D are respectively corresponding to the differences of the impedance values of experimental and control groups having the initial cell seeding densities of 5*10⁵ cells/mL, 2.5*10⁵ cells/mL, 1.2*10⁵ cells/mL and 5*10⁴ cells/mL.

Please still refer to FIG. 6, where the differences of the impedance values on curves A, B, C and D are 37Ω, 20Ω, 14Ω and 11Ω respectively. Accordingly, as shown in FIG. 6, it is understood that the higher initial cell seeding density of MG-63 cells are seeded, the more Integrins are expressed and the higher difference of the impedance values is revealed; and it is also shown the increase of the expression of Integrin causing by the proliferation of cells will make the difference of the impedance values become higher.

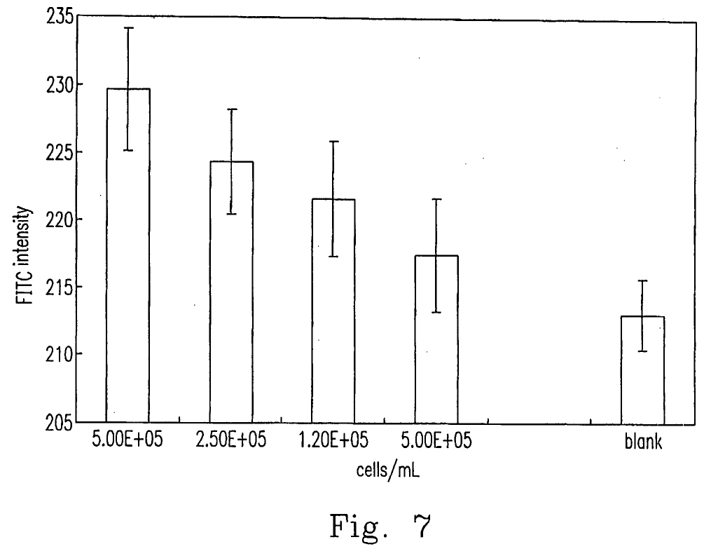

Please refer to FIG. 7, which shows the fluorescein isothiocyanate (FITC) fluorescence intensities of Integrin of MG-63 cells at 48^{th} hour after seeding the cells. Four initial cell seeding densities of MG-63 cells identical to those of experiments revealed in FIG. 6, namely 5*10⁵ cells/mL, 2.5*10⁵ cells/mL, 1.2*10⁵ cells/mL and 5*10⁴ cells/mL, are seeded in 96-wells plate. Then, those MG-63 cells are cultured under the environment and conditions the same as those of FIG. 6's relevant experiments. The X-axis of FIG. 7 shows the initial cell seeding density and the unit thereof is cells/mL, and the Y-axis of FIG. 7 shows the FITC fluorescence intensity. At 48^{th} hour after seeding MG-63 cells, the respective FITC fluorescence intensities of groups of 5*10⁵ cells/mL, 2.5*10⁵ cells/mL, 1.2*10⁵ cells/mL and 5*10⁴ cells/mL are 229.65±4.52, 224.39±3.93, 221.64±4.33 and 217.51±4.20. As shown in FIG. 7, it is known that the higher initial cell seeding density of MG-63 cells are seeded, the more Integrins are expressed.

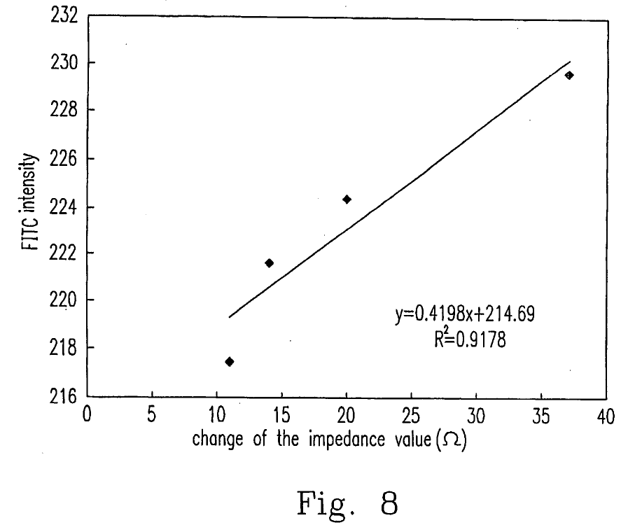

Please refer to FIG. 8, which shows an regression analysis of the relationship between the differences of the impedance values as shown in FIG. 6 and the FITC fluorescence intensities as shown in FIG. 7. The X-axis of FIG. 8 shows the difference of impedance values and the unit thereof is the ohm (Ω), and the Y-axis of FIG. 8 shows the FITC fluorescence intensity. As revealed in FIG. 8, it is known that, under one initial cell seeding density, the difference of impedance values, which is obtained at 48^{th} hour after MG-63 cells being seeded and by apparatus 03, and the FITC fluorescence intensity, which is obtained at 48^{th} hour after MG-63 cells being seeded, have a good linear correlation (R²=0.9178 and p<0.005). Moreover, it is also known from FIG. 8 that the difference of impedance values is in direct proportion with the expression of Integrin. Based on the illustrations of FIG. 8, it is proved ITO electrode chip 01 of the present invention is suitable for continuously and nondestructively observing the expression of the target cellular protein by appropriate antibodies, which is a novel and excellent method for studying and investigating the protein and it expression.

In all of the mentioned experiments, the capacitive reactance value and the resistance value of the ITO electrode are also the appropriate material and indicator for being converted to obtain the data such as the protein expression and the cell numbers.

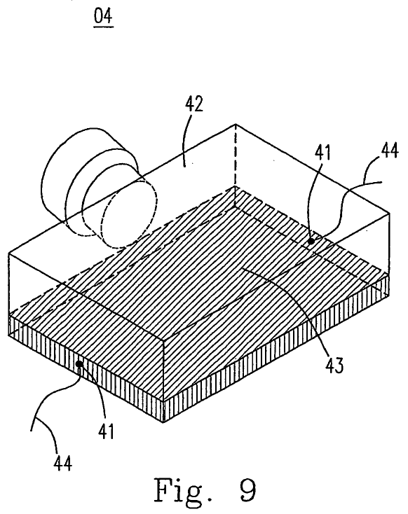

Please refer to FIG. 9, which is a diagram showing a cell culture flask 04 for obtaining the results as shown in FIG. 4 by the suspended cell number calculating method of the present invention. Flask 04 includes a pair of electrodes 41, a body 42, a culture medium 43 and wires 44, wherein electrodes 41 are respectively embedded into two side walls, preferable two oppositional ones, of body 42 and electrically connects both to culture medium 43 in flask 04 and to wires 44, and body 42 is made of an insulating material. In addition, if flask 04 is used for calculating the cell number or observing the growth of the suspended cells by the method of the present invention, culture medium 43 is the serum medium.

Flask 04 is applicable to be substituted for ITO electrode chip 01 included in apparatus 03 as shown in FIG. 3 and electrically connects to other units of apparatus 03 through wires 44. Thus, flask 04 becomes an appropriate device being used in the method of the present invention. Certainly, flask 04 is also suitable for observing and measuring the expression of a target cellular protein. Moreover, flask 04 of the present invention can easily be obtained from modifying a conventional one. In other words, the cost of devices used in the method and/or apparatus for observing and measuring the cell number and the expression of the target protein of the present invention are cheap, but the procedures of continuously observing the cell growth and the expression of a specific cellular protein are apparently improved thereby. Again, the advantages of the present invention are proved.

Based on the above illustrations, the method and apparatus are surly achieving the purpose that performs an automatic, continuous and long-term observation and measurement to a sample, e.g. cells or a specific cellular protein or molecular. The method for detecting/measuring the expression of cellular protein, different from the conventional protein quantitation method such as the immunostaining method, needs no microscope. Moreover, by the continuous and nondestructive method for observing the protein expression of the present invention, both the experimental cost and the human carelessness and/or error are decreased.

While the invention has been described in terms of what are presently considered to be the most practical and preferred embodiments, it is to be understood that the invention need not be limited to the disclosed embodiment. On the contrary, it is intended to cover various modifications and similar arrangements included within the spirit and scope of the appended claims, which are to be accorded with the broadest interpretation so as to encompass all such modifications and similar structures. Therefore, the above description and illustration should not be taken as limiting the scope of the present invention which is defined by the appended claims.

What is claimed is:

1. A method for continuously measuring a protein expression of a cell, comprising steps of:
(a) culturing the cell on an indium tin oxide (ITO) electrode with a medium;
(b) adding a first antibody specifically bound to a protein of the cell into the medium;
(c) adding a second antibody conjugated with a metal particle and specifically bound to the first antibody into the medium;
(d) causing a current flowing through the ITO electrode;
(e) measuring an impedance value of the ITO electrode; and
(f) converting the impedance value into a quantity of the protein expression by a first algorithm.

2. A method according to claim 1, wherein the cell cultured in the step (a) comprises plural kinds of cells.

3. A method according to claim 1, wherein the first antibody comprises a plurality of first antibodies respectively specifically bound to a plurality of proteins of the cell, and the second antibody comprises a plurality of second antibodies respectively specifically bound to the plurality of first antibodies.

4. A method according to claim 1, wherein the current intermittently flows through the ITO electrode.

5. A method according to claim 1, wherein the step (e) is measuring one of a capacitive reactance value and a resistance value of the ITO electrode and the step (f) is converting one of the measured capacitive reactance value and the measured resistance value into the quantity of the protein expression by a second algorithm.

6. An apparatus for measuring a protein expression, comprising:
a cell;
a medium culturing the cell, and having a binder conjugated with a metal particle and specifically bound to a protein of the cell;
an electrode electrically connected with the medium;
a power source electrically connected with the electrode and providing a current flowing through the electrode; and
a measuring unit electrically connected with the electrode and the power source, measuring a change of an impedance value of the electrode and converting the change of the impedance value of the electrode into a quantity of the protein expression by an algorithm.

7. An apparatus according to claim 6, wherein the electrode comprises two wire electrodes, each of which has a width of 0.4 mm.

8. An apparatus according to claim 7, wherein the two wire electrodes are separated from each other by a width of 4 mm.

9. An apparatus according to claim 6, wherein the electrode is made of an indium tin oxide (ITO).

10. An apparatus according to claim 6, wherein the electrode is disposed on a substrate made of one selected from a group consisting of a glass, a quartz, a plastic and a combination thereof.

11. An apparatus according to claim 6, wherein the binder comprises a first antibody and a second antibody conjugated with the metal particle and specifically bound to the first antibody, and the metal particle is a gold particle.

12. An apparatus according to claim 6, wherein the electrode is made in an array.

13. An apparatus according to claim 6, wherein the current is an alternating current.

14. An apparatus according to claim 6, wherein the apparatus further comprises a signal amplifier amplifying the change of the impedance value of the electrode.

15. A method for continuously measuring a protein expression of a cell, comprising steps of:
(a) culturing the cell on an electrode with a medium;
(b) adding a binder conjugated with a metal particle and specifically bound to a protein of the cell into the medium;
(c) causing a current flowing through the electrode;
(d) measuring an impedance value of the electrode; and
(e) converting the impedance value into a quantity of protein expression.

16. A method according to claim 15, wherein the cell in step (a) is cultured on an indium tin oxide (ITO) electrode.

17. A measuring method, comprising steps of:
(a) culturing the cell on an electrode;
(b) causing a current flowing through the electrode;
(c) measuring a first parameter of the electrode; and
(d) converting the first parameter into a second parameter of the cell.

18. A method according to claim 17, wherein the first parameter is an impedance value, the second parameter is a cell number, and the measuring method is used for continuously estimating the cell number.

19. A method according to claim 18, wherein the cell is a suspending cell and is cultured in a serum.

20. A method according to claim 17, wherein the first parameter is converted into the second parameter by an algorithm.
